(19) United States
(12) Patent Application Publication
(10) Pub. No.: US 2023/0015540 A1
(43) Pub. Date: Jan. 19, 2023

(54) Unmanned Flying Vaccine Administration System

(71) Applicants: Cindy Jingru Wang, Palo Alto, CA (US); Connie Ping Jiang, Palo Alto, CA (US); Grace Hui-Ying Li, Palo Alto, CA (US)

(72) Inventors: Cindy Jingru Wang, Palo Alto, CA (US); Connie Ping Jiang, Palo Alto, CA (US); Grace Hui-Ying Li, Palo Alto, CA (US)

(21) Appl. No.: 17/583,587

(22) Filed: Jan. 25, 2022

Related U.S. Application Data
(60) Provisional application No. 63/221,652, filed on Jul. 14, 2021.

(51) Int. Cl. B64C 39/02; G05D 1/10; A61K 39/12

(52) U.S. Cl. CPC B64C 39/024; A61K 39/12; G05D 1/101; B64C 2201/027; B64C 2201/108; B64C 2201/128; B64C 2201/145; B64C 2201/146

(57) ABSTRACT

The unmanned flying vaccine administration system comprises a drone, a vaccine delivery system, an interaction system. The drone is a vaccine injection flying robot that avoids the dangers of in-person vaccination. The vaccine delivery system is an electronic system that harnesses the power of technology to vaccinate people safely and efficiently. The interaction system is an electronic system armed with an Artificial Intelligence infrastructure. The present invention gathers energy by solar power, administers vaccines with a vaccine injection arm, and properly stores vaccines at the desired temperature with a storage container. The computing device controls the main modules that are designed for vaccine delivery and administration. The interaction system has a patient interface camera, a patient interface display, and a temperature sensor that monitor the state of the patient after receiving a vaccination to ensure the health and safety of the patient.

### FIELD OF THE INVENTION

The present invention relates generally to a system for facilitating vaccine administration using a flying vaccine robot. Further, the present invention allows for vaccine administration without person to person contact to aid the distribution efficiency of vaccinations.

### BACKGROUND OF THE INVENTION

In 2020, the COVID-19 virus spread across the globe unexpectedly, creating significant supply and demand constraints in various aspects of human life. Researchers and scientists quickly responded, culminating in the first COVID-19 vaccine roll-out in the US on Dec. 15, 2020. With the completion of effective COVID-19 vaccines, demand from citizens skyrocketed. Currently, there exists a shortage of medical personnel to distribute the vaccine timely. Much of the globe has fallen into a biting quagmire: governments have stockpiles of vaccines but not the means of effectively distributing them. The present invention intents to combine a vaccine administration system with a drone, that is an unmanned flying vehicle. This utilization of technology allows for the present invention to both deliver and administer vaccines robotically without requiring person to person interactions or large vaccination sites.

### BRIEF DESCRIPTION OF THE DRAWINGS

FIG. 1 is an isometric perspective view of the present invention.

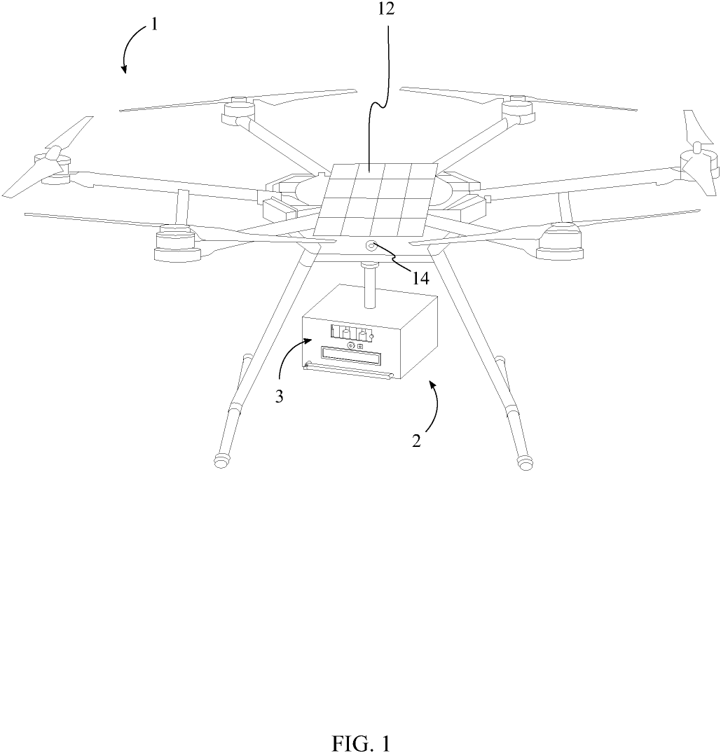

FIG. 2 is a perspective view of the vaccine delivery system and interaction system.

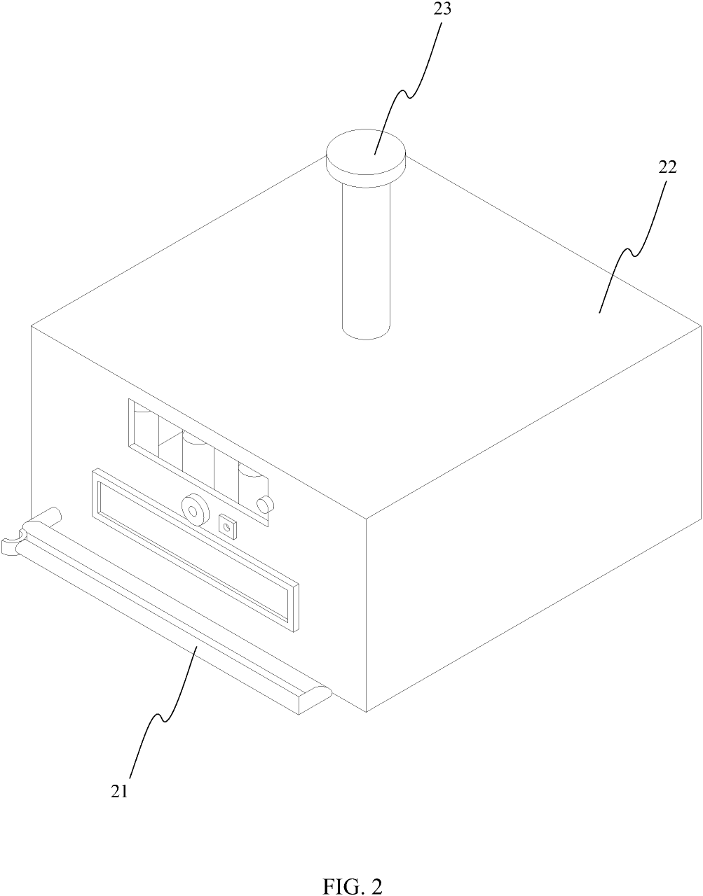

FIG. 3 is a front view of the vaccine delivery system and interaction system.

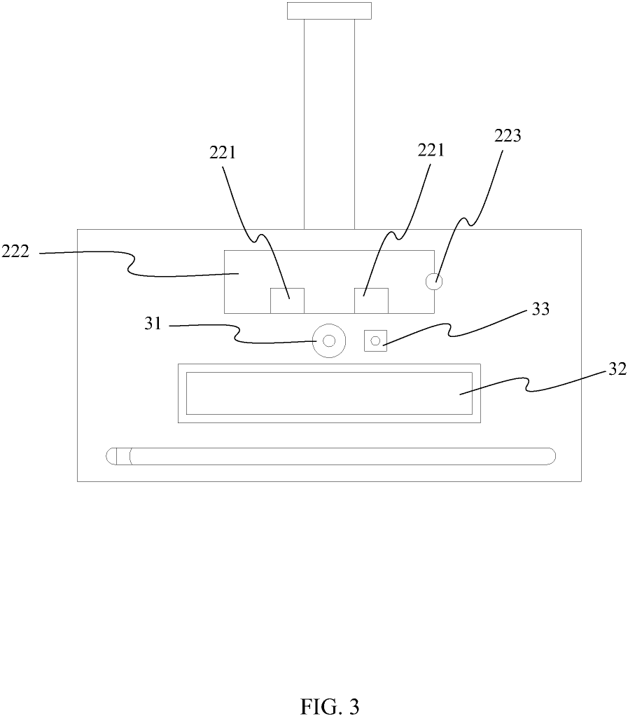

FIG. 4 is a left-side view of the vaccine delivery system and interaction system.

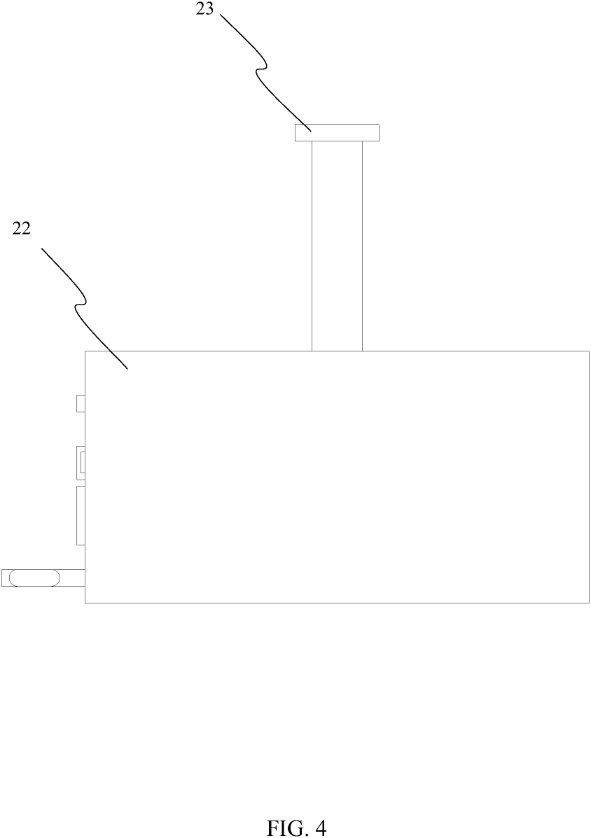

FIG. 5 is a top view of the vaccine delivery system and interaction system.

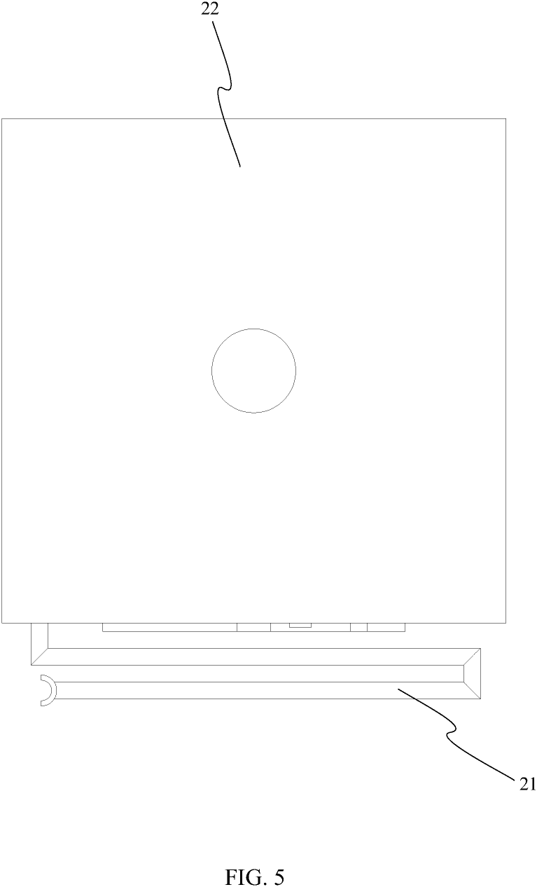

FIG. 6 is a box diagram of the drone.

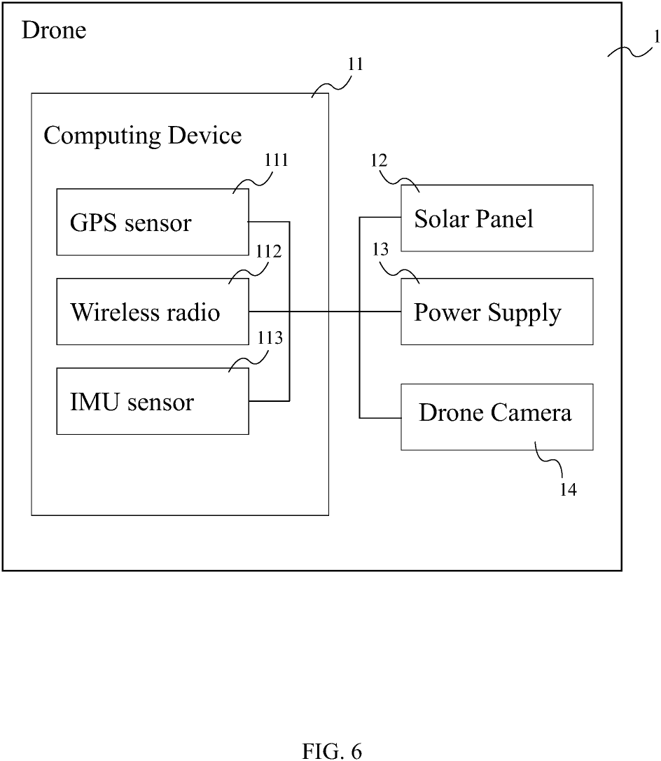

FIG. 7 is a flow chart of the method of operation of the present invention.

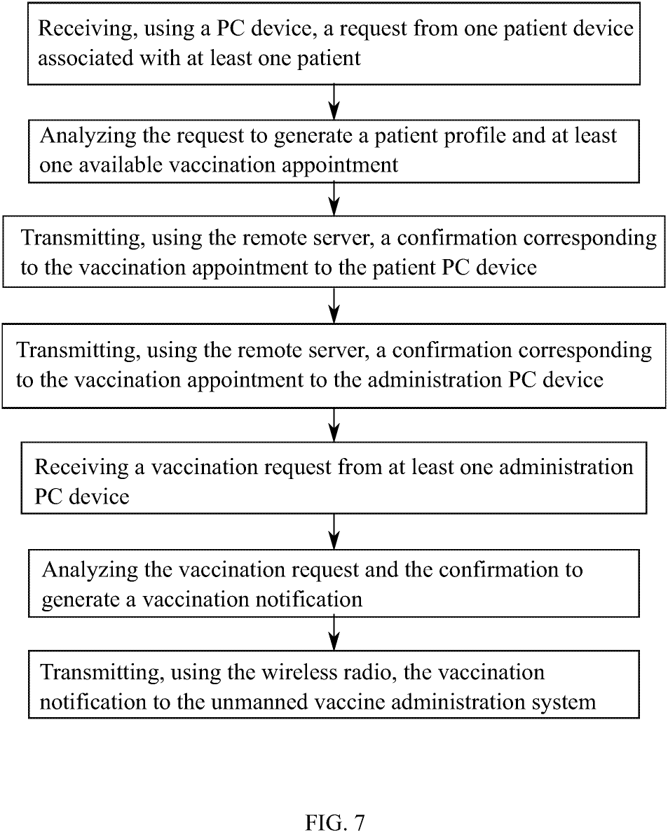

FIG. 8 is a flow chart of the method of operation of the present invention.

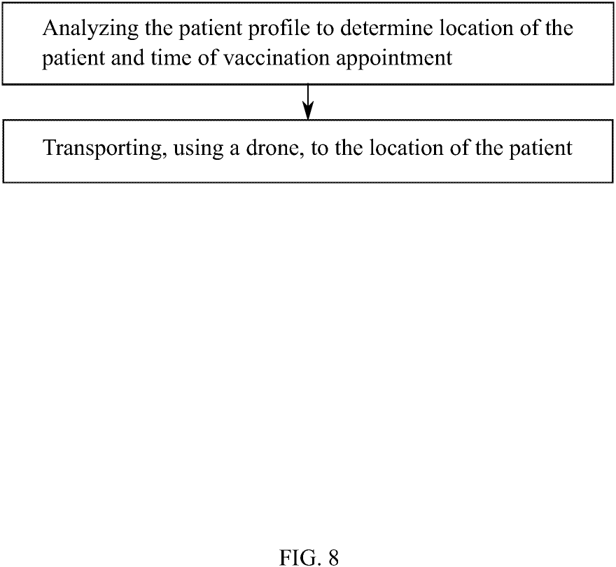

FIG. 9 is a flow chart of the method of operation of the present invention.

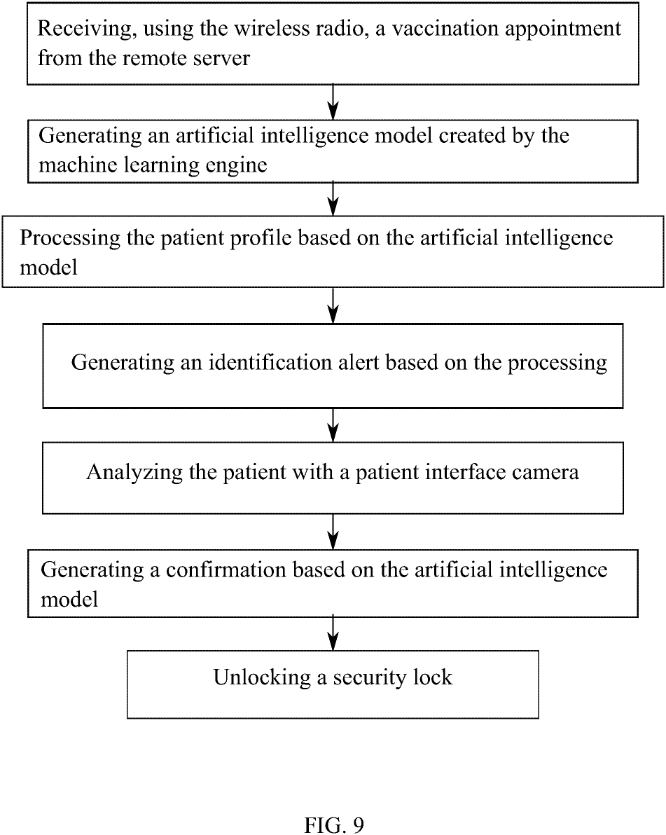

FIG. 10 is a flow chart of the method of operation of the present invention.

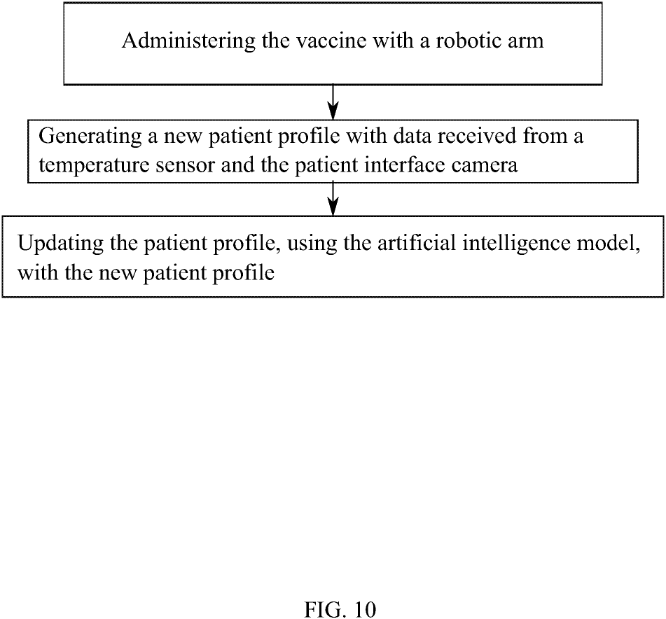

FIG. 11 is a flow chart of the method of operation of the present invention.

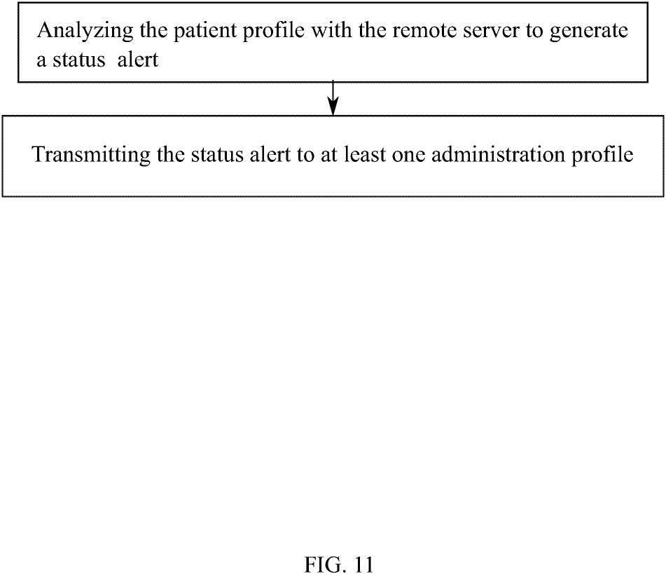

### DETAIL DESCRIPTIONS OF THE INVENTION

All illustrations of the drawings are for the purpose of describing selected versions of the present invention and are not intended to limit the scope of the present invention.

The preferred embodiment of the present invention comprises a drone 1, a vaccine delivery system 2, an interaction system 3. The drone 1 is a flying unmanned vehicle that can travel to various locations by remote or automated control. The vaccine delivery system 2 is an electronic system intended to identify a patient and administer a vaccine to said patient. The interaction system 3 is an electronic system intended to provide and receive feedback from the patient. The drone 1 comprises a computing device 11, a solar panel 12, a power supply 13 and a drone camera 14. The computing device 11 controls the electronic components within the present invention. The vaccine delivery system 2 comprises a robotic arm 21, a storage container 22, and a fastener 23. The robotic arm 21 is an electronic device designed to remove a vaccine from the storage container 22 and administer it to the patient. The fastener 23 is a mechanical device that secures the storage container 22 to the drone 1. Specifically, embodiments of the present invention include at least one fastener 23 selected from the group comprising magnets, clamps, clips, and screws. The interaction system 3 comprises a patient interface camera 31, a patient interface display 32, and a temperature sensor 33. The patient interface camera 31 is an electronic device designed to gather feedback responses from the patient and to monitor the patient’s body. The patient interface display 32 is an electronic device that sends and receives data between the patient and the present invention. The vaccine delivery system 2 is mounted offset the drone 1. As a result, the vaccine delivery system 2 can be utilized once the drone 1 has landed or while still in flight. The interaction system 3 is mounted offset the vaccine delivery system 2. Consequently, the interaction system 3 is able to receive and send data to the patient. The computing device 11 is integrated within the drone 1. Accordingly, the computing device 11 is able to control the flight path and other operations of the drone 1. The computing device 11 is electronically connected to the vaccine delivery system 2 and the interaction system 3. Thus, the computing device 11 is able to control the vaccine delivery system 2 and interaction system 3. The solar panel 12 is mounted on the drone 1, opposite the vaccine delivery system 2. So, the solar panel 12 can receive solar rays to be converted into electrical power for the present invention. The power supply 13 is integrated within the drone 1. As a result, the power supply 13 is able to provide electrical power to the various components of the present invention. The drone camera 14 is integrated into a lateral sidewall of the drone 1. Consequently, the drone camera 14 is able to gather visual information about the present invention’s surroundings. The drone camera 14 is electronically connected to the computing device 11. Accordingly, the drone is able to send visual information to the computing device 11. The drone camera 14, solar panel 12, and computing device 11 are electrically connected to the power supply 13. Thus, the drone camera 14, solar panel 12, and computing device 11 all receive electrical power from the power supply 13. The robotic arm 21 is mounted on the vaccine delivery system 2. So, the robotic arm 21 is able to administer a vaccine to the patient. The storage container 22 is terminally connected to the vaccine delivery system 2, opposite the drone 1. As a result, the storage container 22 remains easily accessible, ensuring convenient vaccine administration. The fastener 23 is terminally connected to the vaccine delivery system 2, adjacent to the drone 1. Consequently, the fastener 23 allows the vaccine delivery system 2 to conveniently attach to the drone 1 and detach from the drone 1. The robotic arm 21 and storage container 22 is electrically connected to the computing device 11 of the drone 1. Accordingly, the robotic arm 21 and storage container 22 are mechanically controlled by the computing device 11. The patient interface camera 31 is integrated into a lateral sidewall of the interaction system 3. Thus, the patient interface camera 31 is able to receive visual information about the patient. The patient interface display 32 is integrated into a lateral sidewall of the interaction system 3, offset below the interface camera. So, the patient interface display 32 is able to send and receive information between the patient and the present invention without interfering with the patient interface camera 31. The temperature sensor 33 is integrated into a lateral sidewall of the interaction system 3, adjacent to the interface camera. As a result, the temperature sensor 33 monitors the temperature of the patient after receiving the vaccination. The patient interface camera 31, the patient interface display 32 and the temperature sensor 33 are electronically connected to the computing device 11. Consequently, the patient interface camera 31, the patient interface display 32, and the temperature sensor 33 are able to send and receive data with the computing device 11, allowing for an interconnected monitoring system.

Furthermore, the computing device 11 as described in the previous paragraph also comprises a Global Positioning System (GPS) sensor 111, an Inertial Measurement Unit (IMU) sensor 112 and a wireless radio unit 113. The GPS sensor 111 is an electronic device that identifies and monitors the location of the present invention. The IMU sensor 112 is an electronic device that measures the inertial movement and speed of the drone 1 throughout the flight path. The wireless radio unit 113 is an electronic device that allows the present invention to remotely communicate. The GPS sensor 111 is mounted within the computing device 11. As a result, the GPS sensor 111 is protected from external elements that could interfere with the signals being sent to the computing device 11. The IMU sensor 112 is mounted within the computing device 11. Consequently, the IMU sensor 112 is able to be easily connected to the computing device 11. The wireless radio unit 113 is mounted within the computing device 11. Accordingly, the wireless radio unit 113 is protected from external elements that could interfere with the signals being sent and received by the wireless radio unit 113. The GPS sensor 111, IMU sensor 112 and wireless radio unit 113 being electronically connected to the computing device 11. Thus, the GPS sensor 111, IMU sensor 112 and wireless radio unit 113 is able to send and receive electronic signals with the computing device 11, creating an interconnected circuit between all components.

Additionally, the present invention comprises a storage container 22 for storing vaccinations. The storage container 22 further comprises at least one vial 221, an exit window 222, and a security lock 223. The vial is a medical tube designed to contain the desired vaccine to be administered. The exit window 222 is an opening on the storage container 22 to allow at least one vial 221 to be removed from the storage container 22. The security lock 223 is a mechanical device that is electronically controlled to secure the storage container 22 only allowing a vaccine to be removed when authorized. At least one vial 221 is mounted within the storage container 22. As a result, the vial is stored within the temperature-controlled storage container 22 until the vaccine is ready to be administer to a patient. The exit window 222 is integrated onto the lateral sidewall of the storage container 22. Consequently, the exit window 222 can be opened allowing the robotic arm 21 to remove a vial from the storage container 22. The security lock 223 is mounted adjacent the exit window 222. Accordingly, the security lock 223 keeps the storage container 22 locked until a vaccine removal has been authorized.

The present invention also pertains to a method of operating an unmanned flying vaccine administration system. The system for executing the method of the present invention provides at least one vaccination appointments stored on at least one remote server. Each vaccination appointment refers to a scheduled time slot selected by the patient where the unmanned flying vaccine administration system travels to the patient to administer a vaccination. The term “remote server” is used herein to refer to a computing device 11 capable of executing all background processes required to perform the method of the present invention. Additionally, the method of the of present invention provides at least one patient profile managed by the remote server, wherein the patient profile is associated to at least one patient personal computing (PC) device. The term “computing device” 11 is used herein to refer to any electronic system capable of executing the method of the present invention and communicating with external devices. In some embodiments, the user employs computing devices 11 selected from the group including, but not limited to, smart phones, smart glasses, tablet computers, and laptop computers. The user profile is a virtual representation of the user and includes user preferences, as well as user identification data. Further, the method of the present invention provides at least one administrative profile managed by at least one health care provider on the remote server, wherein the administration profile is associated to at least one administration PC device. In its preferred embodiment the present invention comprises a drone 1, a vaccine delivery system 2, and an interaction system 3. The PC device receives a request from one patient device associated with at least one patient. As a result, the patient can request a vaccination appointment from any patient device. The method continues by analyzing the request to generate a patient profile and at least one available vaccination appointment. Consequently, the patient profile is created based on the patient information and is provided with potential vaccination appointment time slots. The method continues by transmitting, using the remote server, a confirmation corresponding to the vaccination appointment to the patient PC device. Accordingly, the remote server handles the transfer of information to the patient PC device. The method continues by transmitting, using the remote server, a confirmation corresponding to the vaccination appointment to the administration PC device. Thus, the remote server notifies an administrator of the additional vaccination appointment. The method continues by receiving a vaccination request from at least one administration PC device. So, the administration PC device can manage the approval of vaccination appointments. Afterwards, the method continues by analyzing the vaccination request and the confirmation to generate a vaccination notification. As a result, the vaccination notification is only generated for patients eligible to receive a vaccination approved by an administrator PC device. Finally, the wireless radio unit 113, transmits the vaccination notification to the unmanned vaccine administration system. Consequently, the vaccination notification is sent to the unmanned vaccine administration system to be added to the queue of patients needing a vaccination.

The method of the present invention utilizes machine learning to optimally provide the best experience to the patient. The machine learning achieves this by completing a series of steps by taking the patient records and comparing them to the standard patient record, subjecting the patient to a vaccination, recording an analyzing the patient vaccination response, comparing the analyzed response to the standard vaccination response to create a better standard patient model, sending patient vaccination response to an administrative PC device, and looping back to the first step to repeat the process. The method continues by analyzing the patient profile to determine location of the patient and time of vaccination appointment. As a result, the present invention creates a destination end point linked with the patient profile with a vaccination appointment time. The method finishes by transporting, using a drone 1, to the location of the patient. Consequently, the drone 1 travels to the specified location at the time of the confirmed vaccination appointment.

The vaccination appointment is stored on the remote server. The method continues by receiving, using the wireless radio unit 113, a vaccination appointment from the remote server. As a result, the present invention receives data on the current vaccination appointment from the remote server. The method continues by generating an artificial intelligence model created by the machine learning engine. Consequently, the artificial intelligence model is created and tailored specifically to the patient receiving a vaccination. The method continues by processing the patient profile based on the artificial intelligence model. Accordingly, the patient profile is compared to indicate a match with the artificial intelligence model created. The method continues by generating an identification alert based on the processing. Thus, the identification alert indicates the identity of the patient. The method continues with analyzing the patient with a patient interface camera 31. So, the patient interface camera 31 monitors the patient identity to ensure the vaccine is being administered to an approved patient. Further, the method continues by generating a confirmation based on the artificial intelligence model. As a result, the identity of the patient has been verified by the artificial intelligence model. The method concludes by unlocking a security lock 223. Consequently, the security lock 223 allows for a vaccine to be removed from the storage container 22.

The method of the present invention allows for administering the vaccine with a robotic arm 21. As a result, the robotic arm 21 automatically provides the patient with the proper vaccine. The method continues with generating a new patient profile with data received from a temperature sensor 33 and the patient interface camera 31. Consequently, the temperature sensor 33 and patient interface camera 31 monitor the patient for negative vaccine response symptoms. The method continues with updating the patient profile, using the artificial intelligence model, with the new patient profile. Accordingly, the patient profile is updated with any vaccine response symptoms.

The method of operating an unmanned flying vaccine administration system constantly monitors the wellbeing of the patient receiving the vaccination. The present invention provides at least one status alert. The status alert is a ranking system dictated by the user indicating the wellbeing of the patient and if they are experiencing any negative vaccination response symptoms. The method continues with analyzing the patient profile with the remote server to generate a status alert. As a result, the status alert compares the status of the patient with their patient profile to find any irregularities between the two sets of data. The method concludes by transmitting the status alert to at least one administration profile. Consequently, the status alert is sent to an administration profile if any irregularities are found, and the patient is experiencing any negative symptoms.

Although the invention has been explained in relation to its preferred embodiment, it is to be understood that many other possible modifications and variations can be made without departing from the spirit and scope of the invention as hereinafter claimed.

What is claimed is:

1. An unmanned flying vaccine administration system comprising:
a drone;
a vaccine delivery system;
an interaction system;
the drone comprising a computing device, a solar panel, a power supply and a drone camera;
the vaccine delivery system comprising a robotic arm, a storage container, and a fastener;
the interaction system comprising a patient interface camera, a patient interface display and a temperature sensor;
the vaccine delivery system being mounted offset the drone;
the interaction system being mounted offset the vaccine delivery system;
the computing device being integrated within the drone;
the computing device being electronically connected to the vaccine delivery system and the interaction system;
the solar panel being mounted on the drone, opposite the vaccine delivery system;
the power supply being integrated within the drone;
the drone camera being integrated into a lateral sidewall of the drone;
the drone camera being electronically connected to the computing device;
the drone camera, solar panel, and computing device being electrically connected to the power supply;
the robotic arm being mounted on the vaccine delivery system;
the storage container being terminally connected to the vaccine delivery system, opposite the drone;
the fastener being terminally connected to the vaccine delivery system, adjacent to the drone;
the robotic arm and storage container being electrically connected to the computing device of the drone;
the patient interface camera being integrated into a lateral sidewall of the interaction system;
the patient interface display being integrated into a lateral sidewall of the interaction system, offset below the interface camera;
the temperature sensor camera being integrated into a lateral sidewall of the interaction system, adjacent to the interface camera; and
the patient interface camera, the patient interface display and the temperature sensor being electronically connected to the computing device.

2. The unmanned flying vaccine administration system as claimed in claim 1 comprising:
the computing device further comprising a GPS sensor, a IMU sensor and a wireless radio unit;
the GPS sensor being mounted within the computing device;
the IMU sensor being mounted within the computing device;
the wireless radio unit being mounted within the computing device; and
the GPS sensor, IMU sensor and wireless radio unit being electronically connected to the computing device.

3. The unmanned flying vaccine administration system as claimed in claim 1 comprising:
the storage container further comprising at least one vial, an exit window, and a security lock;
at least one vial being mounted within the storage container;
the exit window being integrated onto the lateral sidewall of the storage container; and
the security lock being mounted adjacent the exit window.
